(19) United States
(12) Patent Application Publication
(10) Pub. No.: US 2007/0240715 A1
(43) Pub. Date: Oct. 18, 2007

(54) Hot air extraction system

(76) Inventor: Markuss Hill, Azusa, CA (US)

Correspondence Address: MARKUSS HILL, 792 N. TODD AVE., AZUSA, CA, 91702-2227, US

(21) Appl. No.: 11/402,430

(22) Filed: Apr. 12, 2006

(51) Int. Cl. A61M 15/00; A61M 16/00

(52) U.S. Cl. 128/203.27; 128/203.15; 128/203.26

(57) ABSTRACT

A hand-held electrical apparatus (the “vaporizer”) for vaporizing substances with a separate, hand-held substance receptacle/device (the “pipe”) through which vapors may be inhaled. The vaporizer is comprised of a backlit, electrically-powered heating element that is enclosed inside two concentric transparent glass tubes. One end of the pipe is designed to mate with the illuminated glass element enclosure on the vaporizer. In other words, the glass tube on the vaporizer fits snugly inside the substance receptacle end of the pipe to allow for Direct Contact Visual Vaporization. While the pipe is mated to the vaporizer, air is sucked into the separate filtered air intake on the side of the housing and passes through the illuminated glass element enclosure where it is heated by the electrical element. Then, the heated air is introduced to the substance in the receptacle, and vapors are produced. These vapors then continue flowing through the pipe and into the user's mouth.

### BACKGROUND OF THE INVENTION

1. Field of the Invention

This invention relates generally to the field of apparatus for an electric vaporizer. Moreover it pertains specifically to such apparatus for a hand-held device for vaporizing various substances with a separate, hand-held substance receptacle/device through which vapors may be inhaled.

2. Description of Related Art

In my past searches in the marketplace for a vaporizer that is altogether efficient, user friendly, and portable, I found many designs available that achieved some, but not all of these criteria In the case of asthma sufferers, portability and user friendly operation are issues of paramount importance. The Storz application (U.S. Pat. No. 6,513,524) is one such example which lacks these important aspects. As a table-top vaporizer, it lacks portability, and its temperature-control mechanism is hardly user friendly.

Also of note is the Wheeler application (U.S. Pat. No. 5,993,748). Although it performs well as a vaporizer as does the Storz model, its inherent design flaw is the position of the intake holes and the heating element such that device cleaning becomes very difficult. Hence, I have designed a better system that overcomes these and other drawbacks of the prior art.

### SUMMARY OF THE INVENTION

In view of the limitations now present in the prior art, the present invention provides a new and useful Hot Air Extraction System which is simpler in construction, more universally usable and more versatile in operation than known apparatus of this kind.

The purpose of the present invention is to provide a new Hot Air Extraction device that has many novel features not offered by the prior art apparatus that result in a new Hot Air Extraction System which is not apparent, obvious, or suggested, either directly or indirectly by any of the prior art apparatus.

The present invention is a system for extracting vapors from various substances that also provides for the inhalation of such vapors. The system is comprised of two separate, hand-held devices: the vaporizer and the pipe.

The vaporizer is comprised of an electrically powered heating element that is enclosed inside a transparent glass tube. Inside this exterior glass tube is a separate, smaller diameter glass tube, around which a metal wire has been circumferentially wound from top to bottom Together, these two concentrically aligned glass tubes and wound metal wire comprise our unique electrically powered heating element. One end of the exterior tube attaches to a base block which is hidden within the vaporizer's housing, and has an orifice to connect this end of the glass tube to the filtered air intake which is mounted to the side of the vaporizer housing. Mounted directly behind the base block within the vaporizer housing and perpendicular to the glass tubes is a small light source which serves to illuminate the entire length of both glass tubes. Surrounding the exterior glass tube is a heat shield which is fastened directly to the vaporizer housing. It consists of three discs that are separated by spacers to provide for both air flow/cooling of glass element enclosure and visibility of internal components. The heat shield also protects the illuminated glass element enclosure from breakage.

On the housing, mounted above the glass tube and heat shield is a 3-way rotating switch that allows for different power settings. Attached on the top side of the vaporizer housing is the handle which contains the electrical wiring that connects to either a transformer-plug or a 12V plug adapter. The electrical wires, connections, base block, and all other internal components are housed within a rectangular aluminum box that has a removable back-plate for access.

The pipe functions as a receptacle for the substance to be vaporized as well as a device for the inhalation of the vapors. It is made of glass. One end is designed to mate with the illuminated glass element enclosure on the vaporizer. In other words, the glass tube on the vaporizer fits snugly inside the substance receptacle end of the pipe. The other end of the pipe is fashioned to fit comfortably between the user's lips for the purpose of inhalation of the vapors. Air flow through the vaporizer and pipe is provided by user inhalation. While the pipe is mated to the vaporizer, air is sucked into the filtered air intake on the side of the housing and passes through the illuminated glass element enclosure where it is heated by the electrical element. Then, the heated air is introduced to the substance in the receptacle, and vapors are produced. These vapors then continue flowing through the pipe and into the user's mouth.

The foregoing has outlined, in general, the physical aspects of the invention and is to serve as an aid to better understanding the more complete detailed description which is to follow. In reference to such, there is to be a clear understanding that the present invention is not limited to the method or detail of construction, fabrication, material, or application of use described and illustrated herein. Any other variation of fabrication, use, or application should be considered apparent as an alternative embodiment of the present invention.

### BRIEF DESCRIPTION OF THE DRAWINGS

The following drawings further describe by illustration the advantages and objects of the present invention. Each drawing is referenced by corresponding figure reference characters within the “DETAILED DESCRIPTION OF THE INVENTION” section to follow.

FIG. 1. is a perspective view of a two-piece system for vaporizing substances that provides for the efficient, convenient inhalation of vapors according to the present invention.

FIG. 2. is a side view of a two-piece system for vaporizing substances that provides for the efficient, convenient inhalation of vapors according to the present invention.

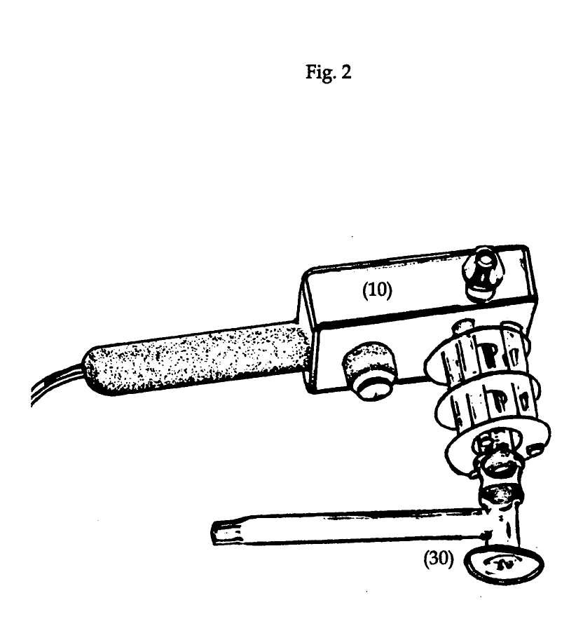

FIG. 3. is a side view of a substance receptacle/vapor inhalation device or “pipe” according to the present invention, shown in the grasp of a user's hand.

FIG. 4. is an exploded sectional view of a two-piece system for vaporizing substances that provides for the efficient, convenient inhalation of vapors according to the present invention.

FIG. 5. is a side view of 2 types of power sources used according to the present invention.

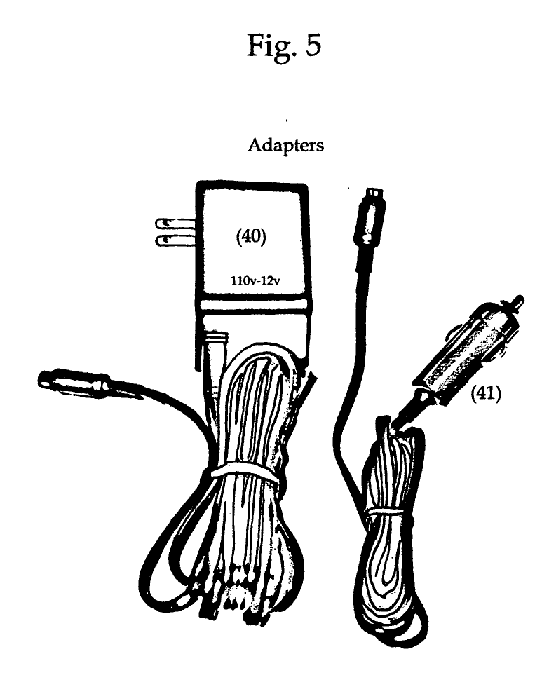

### DETAILED DESCRIPTION OF THE INVENTION

Referring now descriptively to the drawings, the attached figures illustrate a Hot Air Extraction Apparatus.

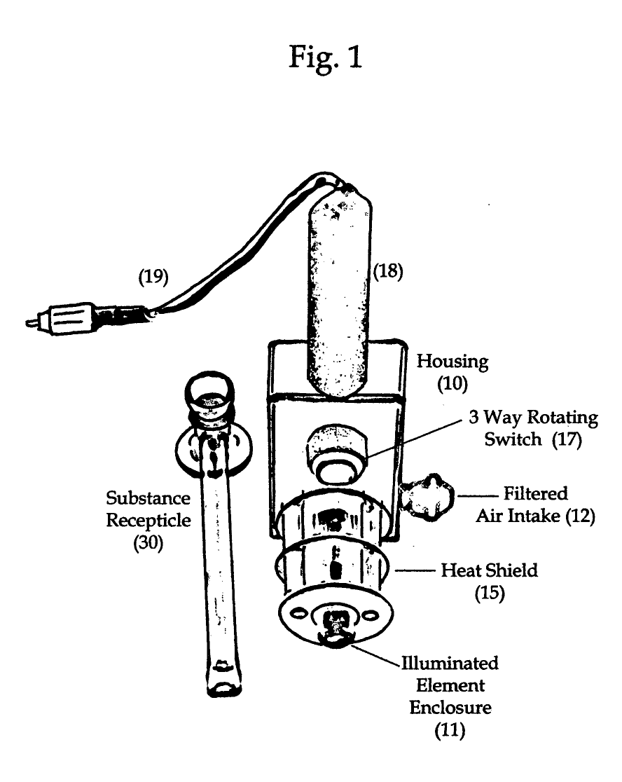

FIGS. 1 illustrates a Hot Air Extraction apparatus according to the present invention, a system for vaporizing various substances that provides for the efficient, convenient inhalation of such vapors.

The present invention is a system for extracting vapors from various substances that also provides for the inhalation of such vapors, as seen in FIG. 1. The system is comprised of two separate, hand-held devices: the vaporizer (10) and the pipe (30).

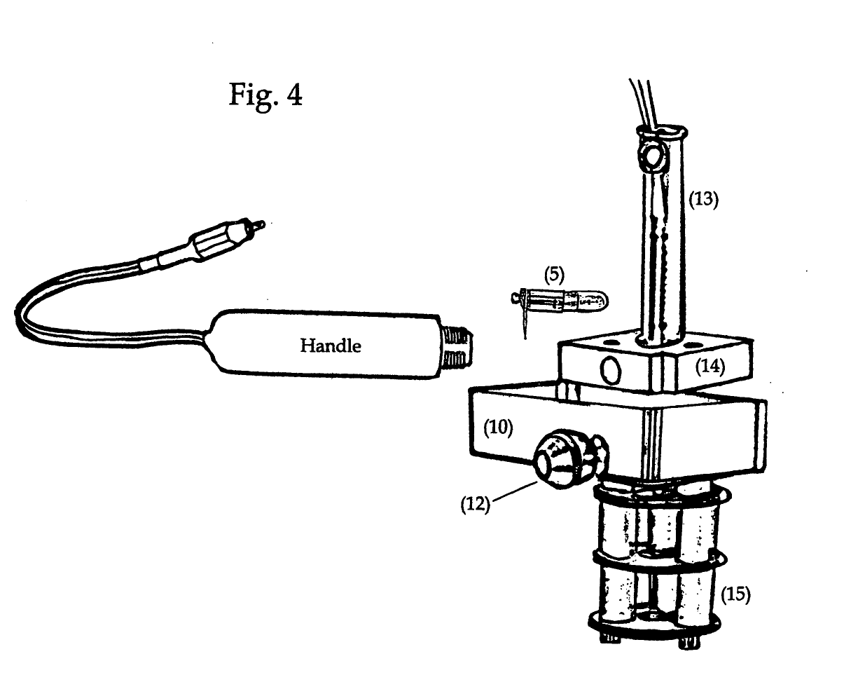

Referring to both FIGS. 1 and 4, the vaporizer (10) is comprised of an electrically powered heating element (13) that is enclosed inside a transparent glass tube (11). One end of this glass tube attaches to a base block (14) which is hidden within the vaporizer's housing, and has an orifice to connect this end of the glass tube to the filtered air intake (12) which is mounted to the side of the vaporizer housing (10). Mounted directly behind the base block within the vaporizer housing and perpendicular to the concentric glass tubes is a small light source (5) which serves to illuminate the entire length of glass tube (11). Surrounding the glass tube is a heat shield (15) which is fastened directly to the vaporizer housing (10). It consists of three discs that are separated by spacers to provide for both air flow/cooling of glass element enclosure and visibility of internal components. The heat shield also protects the illuminated glass element enclosure from breakage.

Mounted above the glass tube and heat shield is a 3-way rotating switch (17) that allows for different power settings. Attached on the top side of the vaporizer housing (10) is the handle (18) which contains the electrical wiring (19) that connects to either a transformer plug (40) or a 12V plug adapter (41). The electrical wires, connections, base block, and all other internal components are housed within a rectangular aluminum box (10) that has a removable back-plate for access.

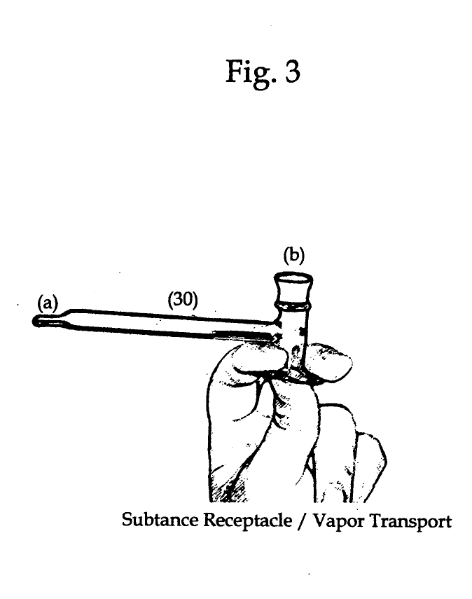

The pipe (30) functions as a receptacle for the substance to be vaporized as well as a device for the inhalation of the vapors. It is made of glass. As shown in FIG. 3, one end (b) is designed to mate with the illuminated glass element enclosure (11) on the vaporizer. In other words, the glass tube (11) on the vaporizer fits snugly inside the substance receptacle end of the pipe (b). Also shown in FIG. 3, the other end of the pipe (a) is fashioned to fit comfortably between the user's lips for the purpose of inhalation of the vapors. Air flow through the vaporizer and pipe is provided by user inhalation. While the pipe (30) is mated to the vaporizer (10), air is sucked into the filtered air intake (12) on the side of the housing and passes through the illuminated glass element enclosure (11) where it is heated by the electrical element (13). Then, the heated air is introduced to the substance in the receptacle (b), and vapors are produced. These vapors then continue flowing through the pipe (30) and into the user's mouth.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

### OBJECTS OF THE INVENTION

Accordingly several advantages and objects of the present invention are:

A principal object of the present invention is to provide a new system for extracting vapors from substances as well as providing for the efficient, convenient inhalation of such vapors, and that will overcome the deficiencies of the prior art devices.

An object of the present invention is to provide a hot air extraction device that provides the convenience of portability with a unique, eye-level, variable right-angle posture of use.

Another object of the present invention is to provide a hot air extraction device that is equipped for use with a 12V adapter and a standard two-prong 110V electrical plug, thus allowing for use with DC as well as AC power sources.

Another object of the present invention is to provide a hot air extraction device that is equipped with a separate, filtered air intake.

Another object of the present invention is to provide a hot air extraction device that is equipped with a unique, illuminated transparent element enclosure.

Another object of the present invention is to provide a hot air extraction device that is equipped with a hi-lo-off switch to vary the heat settings and thus allow for the vaporization of a broad range of substances.

Another object of the present invention is to provide a hot air extraction device that is comprised of a two-piece system (vaporizer and pipe) which keeps the substance to be vaporized away from electrical connections and heating surfaces, thus promoting a longer service life for the vaporizer. The pipe is a hand-held combination receptacle for the substance to be vaporized and mouthpiece for oral inhalation.

Another object of the present invention is to provide a hot air extraction device that is more universally functional in today's market than the prior art devices.

It is intended that any other advantages and objects of the present invention that become apparent or obvious from the detailed description or illustrations contained herein are within the scope of the present invention.

What is claimed:

1. A system for the vaporization and inhalation of various substances that is comprised of:
a housing;
an electrically powered heating element within said housing that consists of two concentrically-aligned transparent glass tubes, the inner of which has been wound circumferentially with metal wire;
a separate, filtered air intake mounted to said housing;
a pipe that acts as both substance receptacle and vapor inhalation device, which is designed to encompass and form a glass-glass seal with exterior glass tube of said heating element.

2. A device according to claim 1, wherein a small light source is mounted directly behind and perpendicular to said glass tubes within said housing to provide illumination of entire length of glass element enclosure.

3. A device according to claim 1, wherein a multiple-position power switch is attached to said housing to vary the temperature of said heating element.

4. A device according to claim 1, wherein a modular provision has been made in the electrical wiring to allow for quick, easy interchangeability between either AC or DC power sources.
